(19) United States
(12) Patent Application Publication
(10) Pub. No.: US 2007/0190139 A1
(43) Pub. Date: Aug. 16, 2007

(54) Delayed release pharmaceutical oral dosage form and method of making same

(75) Inventors: Horst G. Zerbe, Hudson (CA); Pompilia Ispas-Szabo, Greenfield Park (CA)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: Intelgenx Corp., Saint-Laurent (CA)

(21) Appl. No.: 11/403,262

(22) Filed: Apr. 13, 2006

Related U.S. Application Data
(60) Provisional application No. 60/772,547, filed on Feb. 13, 2006.

(51) Int. Cl. A61K 9/22; A61K 31/557

(52) U.S. Cl. 424/468; 514/573

(57) ABSTRACT

The present invention relates to a multi layer pharmaceutical oral dosage form having delayed release and immediate release properties and method of making same. The delayed release formulation substantially behaves as an enterically coated dosage form but without the formulation and the application of an enteric coating. The delayed release formulation is characterized by a mixture of one or more active ingredients and one or more excipients selected from the group of solid aliphatic alcohols, mixtures of esters of saturated fatty alcohols and saturated fatty acids, natural or synthetic waxes, hydrogenated castor oil, hydrogenated vegetable oil, gums, and mixtures thereof; pH dependent soluble polymers; and optionally an opacifying agent.

This application claims priority to U.S. Provisional Application No. 60/772,547 filed on Feb. 13, 2006 which is herein incorporated by reference in its entirety.

### FIELD OF THE INVENTION

The present invention relates to a delayed release pharmaceutical oral dosage form and method of making same. More specifically, but not exclusively, the present invention relates to a delayed release pharmaceutical oral dosage form comprising a non-steroidal anti-inflammatory drug (NSAID), a prostaglandin analogue compound, and/or a proton pump inhibitor (PPI) and/or an H₂-blocker.

Methods for manufacture of the delayed release pharmaceutical oral dosage forms and use of the delayed release pharmaceutical oral dosage forms in treating disease are disclosed.

### BACKGROUND OF THE INVENTION

Prostaglandin analogue compounds, such as the ones known under the generic names misoprostol, enoprostil, enisoprost and miraprostal, are orally active PGE₁-analogs with mucosal protective and antisecretory properties. They are mainly used for preventing gastric and duodenal ulcers associated with NSAID treatment. They are commonly administered in separate, single unit dosage form, and sometimes in combination with an NSAID in a fixed dosage form.

Proton pump inhibitors (PPIs) are a class of acid-labile pharmaceutical compounds that block gastric acid secretion pathways. Exemplary proton pump inhibitors include, omeprazole (Prilosec.RTM.), lansoprazole (Prevacid.RTM.), esomeprazole (Nexium.RTM.), rabeprazole (Aciphex.RTM.), pantoprazole (Protonix.RTM.), pariprazole, tenatoprazole, and leminoprazole. The drugs of this class suppress gastrointestinal acid secretion by the specific inhibition of the H⁺/K⁺-ATPase enzyme system (proton pump) at the secretory surface of the gastrointestinal parietal cell. Most proton pump inhibitors are susceptible to acid degradation and, as such, are rapidly destroyed in an acidic pH environment in the stomach. Therefore, proton pump inhibitors are often administered as enteric-coated dosage forms in order to permit release of the drug in the duodenum after having passed through the stomach.

Histamine H₂ receptor blocking agents (referred to herein as H₂-blockers) are a class of drugs which act as antagonists of the histamine H₂ receptor. H₂ Blockers are effective means of inhibiting gastric acid secretion. Such compounds have a delayed onset, generally one to two hours after ingestion, and a long duration of action. Specific, H₂ blockers include cimetidine, ranitidine, ebrotidine, pabutidine, lafutidine, loxtidine and famotidine.

Non-steroidal anti-inflammatory drugs (NSAIDs) are among the most commonly prescribed and used drugs world-wide. The ability of NSAIDs to treat inflammatory disorders is attributed to their ability to inhibit cyclooxygenase, the enzyme responsible for biosyntheses of the prostaglandins and certain autocoid inhibitors, including inhibitors of lipoxygenase and cyclooxygenase (such as cyclooxygenase-I and cyclooxygenase-II). However, despite the therapeutic benefits of NSAIDs, their use is often limited by an increased risk of gastrointestinal side-effects, in particular upper gastrointestinal side-effects such as peptic ulceration, dyspeptic symptoms and risk of bleeding and perforation of the stomach (McGarty D. M., Gastroenterology 1989, 96, 662; Hawkey C, BMJ 1990, 300, 278).

One promising solution to the problem of healing and preventing NSAID associated upper gastrointestinal problems, like ulcers and dyspeptic symptoms in patients needing continuous NSAID treatment, is to avoid contact between the NSAID and acidic gastric juice by delaying the NSAID release or to combine the NSAID treatment with an anti-ulcer drug approved for the healing and/or prophylaxis of NSAID associated gastrointestinal side-effects such as prostaglandin analogues, H₂-blockers, and proton pump inhibitors.

Pharmaceutical oral dosage forms are often better absorbed via the intestine. Delayed release pharmaceutical oral dosage forms can be obtained by applying an enteric film coating on a core tablet containing an active ingredient. Indeed, enteric film coatings have been widely used to allow pharmaceutical oral dosage forms to be released in the intestine rather than the stomach since many pharmaceutical products irritate the stomach due to their chemical properties. Moreover, other pharmaceutical products undergo chemical changes in gastric acid or by the action of stomach or saliva enzymes, thus becoming less effective.

Enteric coatings are generally pH sensitive and will remain essentially impermeable at lower pH so as to pass through the stomach unscathed. Once in the higher pH region of the digestive tract, namely the intestinal tract, the coating will become permeable and allow the release of the active ingredient. Enteric coatings are commonly applied to a compressed tablet core or to individual beads or pellets which are then compressed into a tablet shape or placed in a reservoir capsule. Enteric coatings are widely sold under the trademarks Eudragit® by the Rohm Pharma and exist in various grades.

The procedure of applying an enteric coating to a compressed tablet core generally consists in the preparation of aqueous dispersions/solutions or organic solutions including a polymer, plasticizers, glidants, anti-foam agents, fillers and pigments. The polymer is commonly selected from the class of cellulose derivatives or represents a polymer or copolymer of acrylic and/or methacrylic acid or esters thereof. In the case of the methacrylic polymer class, the coating is usually sprayed onto rotating tablets, pre-warmed to about 40° C., and maintained at a temperature of approximately 40° C. to 50° C. during the entire process. A post-drying step, also known as tablet curing, contributes to film coalescence and improves the film coating characteristics.

The use of enteric coatings, however, involves at least one additional process step, additional costs, and requires skill and know-how in the formulation and application of enteric coatings. Additional excipients such as plasticizers, glidants, anti-foaming agents, fillers and pigments are usually required to obtain suitable properties. For example, the coating must be of appropriate thickness, elasticity, porosity and/or stickiness. All of these parameters introduce additional technical and cost related factors into the manufacturing of enterically coated dosage forms. Various techniques commonly used for the application of enteric coatings include dry coating, spray coating and pan coating.

The prior art on the subject of delayed release is often concerned with non-steroidal anti-inflammatory drug formulations.

U.S. Pat. No. 5,698,225, issued to Gimet et al. on Dec. 16, 1997, proposes a combined NSAID and prostaglandin product. The product is enterically coated and is composed of a core comprising an NSAID selected from diclofenac and piroxicam, which core is surrounded by a mantle coating of a prostaglandin. An intermediate coating can optionally be present between the NSAID core and the prostaglandin mantle coating.

U.S. Pat. No. 6,537,582, issued to Woolfe et al. on Mar. 25, 2003, proposes an oral pharmaceutical dosage form including a mixture of a delayed release formulation of an NSAID and a mixture containing a prostaglandin and one or more excipients. Methods describing the formation of coated granules are disclosed and comprise spraying a coating solution onto a bed of NSAID and any necessary excipients, for example, using a fluid bed coating apparatus. The process is controlled to produce fine granules which do not require milling before incorporation into tablets or capsules. The coating solution may include cellulose derivatives e.g. hydroxypropyl methyl cellulose, methacrylic acid and derivatives (e.g. methyl methacrylates), Eudragit®, especially Eudragit L or S. Normally, the coating will include plasticizers, e.g. polyethylene glycol, triacetin or phthalate esters, conferring the required mechanical characteristics such as flexibility and hardness to the coating.

U.S. Pat. No. 6,365,184, issued to Depui et al. on Apr. 2, 2002, and published U.S. Patent Application 2004/0022846A1 filed by Depui et al. and published on Feb. 5, 2004 describe an oral pharmaceutical dosage form comprising an acid susceptible proton pump inhibitor and one or more NSAIDs in a fixed formulation, wherein the proton pump inhibitor is protected by an enteric coating layer.

U.S. Pat. No. 6,287,600, issued to Ouali et al. on Sep. 11, 2001, discloses a stabilized pharmaceutical composition including an enterically coated NSAID, a prostaglandin and a prostaglandin stabilizing agent. In manufacturing the stabilized pharmaceutical composition, the NSAID was granulated by blending with suitable excipients (i.e. binders, fillers) in a fluid-bed granulator, followed by the application of an enteric coating. The enterically coated NSAID granules were then mixed with a prostaglandin comprising blend and tableted.

As becomes readily apparent from the above, enterically coated systems involve additional process steps and the consideration of additional technical parameters which are time-consuming and which increase manufacturing costs. Coating ingredient selection, dispersion preparations and various technical parameters (i.e. temperature range, droplet size, type and content of plasticizer, etc.) are time-consuming operations and often, for an efficient protection, application of many layers (i.e. undercoatings) is necessary. Enteric coating performance, brittleness and stickiness underline critical shortcomings of enterically coated systems.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation techniques have also been developed in the art and constitute cost efficient, yet, for reasons further described below, seldomly applied processing techniques in the array of pharmaceutical manufacturing operations, including the manufacture of a variety of dosage forms and formulations such as immediate release and sustained release pellets, granules and tablets. Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation can then be provided to a tablet press or be encapsulated for preparing the oral dosage form. Melt granulation improves the dissolution rate and bioavailability of an active (i.e. drug) by forming a solid dispersion or solid solution. However, melt granulation often requires high energy input and cannot be applied to heat-sensitive materials owing to the elevated temperatures involved. Moreover, the use of higher-melting-point binders requires higher melting temperatures which can contribute to instability problems, especially for heat-labile materials. On the up-side, melt granulation provides for a uniform dispersion of the active, involves fewer processing steps, (time consuming drying steps being eliminated), and provides for good stability at varying pH and moisture levels.

U.S. Pat. No. 5,169,645, issued to Shukla et al. on Dec. 8, 1992, discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt. In either case, the melt combination of the wax(es) with the additive(s) yields, upon cooling and granulation, a wax-containing particulate drug diluent having improved and unexpected flow properties.

A great many of the current pharmaceutical products suffer from light induced degradation of active ingredient(s). The traditional approach to remedy this problem has been to provide outer coatings on solid dosage forms, these protective coatings containing opacifying pigments such as titanium dioxide white. Moreover, these coatings commonly comprise polymers and additives to facilitate application and to provide good mechanical resistance. It is also known to mix pigments with active and inactive powder excipients.

Since many patients suffering from inflammatory disorders also suffer from gastric acid related disorders, there remains a need for a novel delayed release pharmaceutical oral dosage form useful for co-administering a NSAID, a prostaglandin analogue compound, and/or a proton pump inhibitor (PPI) and/or an H₂-blocker. Such a novel delayed release pharmaceutical oral dosage form, essentially behaving as an enterically coated dosage form without the need for the formulation and application of an enteric coating, would be a welcome innovation in the pharmaceutical art.

The present invention seeks to meet these and other needs.

The present description refers to a number of documents, the content of which is herein incorporated by reference in their entirety.

### SUMMARY OF THE INVENTION

The present invention relates to a delayed release pharmaceutical oral dosage form that essentially behaves as enterically coated dosage form but without the formulation and the application of an enteric coating. More specifically, as broadly claimed, the present invention relates to a multi-layer pharmaceutical oral dosage comprising a first layer formulated for delayed release of a NSAID and a second layer formulated for immediate release of a prostaglandin analogue compound, and/or a proton pump inhibitor and/or an H₂-blocker.

In an embodiment, the present invention relates to a multi-layer pharmaceutical oral dosage form wherein one or more NSAIDs are essentially uniformly distributed in a composition comprising: one or more excipients selected from the group consisting of solid aliphatic alcohols, mixtures of esters of saturated fatty alcohols and saturated fatty acids, or natural or synthetic waxes, hydrogenated castor oil, hydrogenated vegetable oil, gums, and mixtures thereof, and one or more polymers and/or copolymers exhibiting a pH-dependent solubility. In a further embodiment of the present invention, such a multi-layer delayed release pharmaceutical oral dosage form further comprises at least one type of light opacifying pigment.

In an embodiment, the present invention relates to a multi-layer pharmaceutical oral dosage form wherein one or more NSAIDs are substantially uniformly distributed in a composition comprising: one or more excipients selected from the group consisting of solid aliphatic alcohols, mixtures of esters of saturated fatty alcohols and saturated fatty acids, or natural or synthetic waxes, hydrogenated castor oil, hydrogenated vegetable oil, gums, and mixtures thereof; one or more polymers and/or copolymers exhibiting a pH-dependent solubility; a disintegrant; and light opacifying pigments or flakes in an amount suitable to confer light protective characteristics to the one or more NSAIDs contained in the oral dosage form.

In an embodiment, the present invention relates to a method for manufacturing a multi-layer pharmaceutical oral dosage form. More specifically, as broadly claimed, the present invention relates to a method for manufacturing a multi-layer pharmaceutical oral dosage form comprising formulating a first layer for delayed release of one or more NSAIDs and formulating a second layer for immediate release of a prostaglandin analogue compound, and/or a proton pump inhibitor and/or an H₂-blocker. The first layer, providing for delayed release characteristics, comprises substantially uniformly distributing one or more NSAIDs in a composition comprising: one or more excipients selected from the group consisting of solid aliphatic alcohols, mixtures of esters of saturated fatty alcohols and saturated fatty acids, or natural or synthetic waxes, hydrogenated castor oil, hydrogenated vegetable oil, gums, and mixtures thereof; and one or more polymers and/or copolymers exhibiting a pH-dependent solubility. The second layer, providing for immediate release of the active when exposed to acidic media such as commonly encountered in the gastric region, comprises formulating a prostaglandin analogue compound, and/or a proton pump inhibitor and/or an H₂-blocker with pharmaceutically acceptable excipients using procedures well known to those of ordinary skill in the art. The method of formulating the first layer comprises the steps of obtaining through heating a liquid form of the one or more excipients; mixing the one or more NSAIDs with the one or more polymers and/or copolymers exhibiting a pH-dependent solubility to obtain a blend; and granulating the blend with the liquid form of said one or more excipients so as to obtain granules. More particularly, the granulating step comprises slowly adding in portions the liquid form of the one or more excipients to the blend to obtain a wet mass; and slowly cooling the wet mass at a controlled rate preventing agglomeration to obtain a granulated material.

The foregoing and other objects, advantages and features of the present invention will become more apparent upon reading of the following non restrictive description of illustrative embodiments thereof, given by way of example only with reference to the accompanying drawings.

### BRIEF DESCRIPTION OF THE DRAWINGS

In the appended drawings:

FIG. 1 is a graph illustrating the mixer motor output, the internal bowl temperature and the heating jacket temperature as a function of time for a diclofenac-Na granulation as performed in a 1 L bowl.

FIG. 2 is a graph illustrating the mixer motor output, the internal bowl temperature and the heating jacket temperature as a function of time for a diclofenac-Na granulation scale-up trial as performed in a 6 L bowl, showing homogeneous granules with some fine particles exhibiting excellent flow properties; granules obtained at the latter stages of liquid addition; and granules obtained at the initial 65° C. mixing stage. The leveling off of the mixer motor power output is indicative of the diclofenac-Na being fully coated. As described herein in Example 1, the 6 L bowl speed settings were adjusted in order to obtain an optimal particle size.

FIG. 3 is a graph illustrating the in-vitro release of diclofenac-Na as obtained for tablets prepared according to the formulations of example 2; the graph further illustrates the influence of the type of disintegrant and the proportion thereof on tablet disintegration.

FIG. 4 is a graph illustrating the in-vitro release of diclofenac-Na as obtained for tablets in accordance with Examples 3a, b and c of the present invention (all tablets were kept for two hours in simulated gastric fluid and later transferred to simulated intestinal fluid using a USP apparatus II at 200 rpm).

### DETAILED DESCRIPTION OF ILLUSTRATIVE EMBODIMENTS

In order to provide a clear and consistent understanding of the terms and abbreviations used in the present specification, a number of definitions and abbreviations are provided below. Moreover, unless defined otherwise, all technical and scientific terms as used herein have the same meaning as commonly understood to one of ordinary skill in the art to which this invention pertains.

Abbreviations: SGF: Simulated Gastric Fluid; SIF: Simulated Intestinal Fluid.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or method steps.

The term “about” is used to indicate that a value includes an inherent variation of error for the device or the method being employed to determine the value.

NSAIDs as used in this specification include, but are not limited to aminoarylcarboxylic acid derivatives such as enfenamic acid, etofenamate, flufenamic acid, isonixin, meclofenamic acid, mefenamic acid, niflumic acid, talniflumate, terofenamate, and tolfenamic acid; arylacetic acid derivatives such as aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac isoxepac, lonazolac, metiazinic acid, mofezolac, oxametacine, pirazolac, proglumetacin, sulindac, tiaramide, tolmetin, tropesin, and zomepirac; arylbutyric acid derivatives such as bumadizon, butibufen, fenbufen, xenbucin; arylcarboxylic acids such as clidanac, ketorolac, tinoridine; arylpropionic acid derivatives such as alminoprofen, benoxaprofin, bermoprofen, bucloxic acid, carrageen, fenoprofen, flunoxaprofen, flurbiprofen, ibuprofen, ibuproxam, indoprofen, ketoprofen, loxoprofen, naproxen, oxaprozin, piketoprofin, pirprofen, pranoprofen, protizinic acid, suprofen, tiaprofenic acid, ximoprofen, and zaltoprofen; pyrazoles such as difenamizole and epirozole; pyrazolones such as apazone, benzpiperylon, feprazone, mofebutazone, morazone, oxyphenbutazone, phenylbutazone, pipebuzone, propyphenazone, prostaglandins, ramifenazone, suxibuzone, and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphtyl salicylate, olsalazine, parsalmide, phenyl acetylsalicylate, phenyl salicylate, salacetamide, salicylamide o-acetic acid, salicylsulfuiric acid, salsalate, sulfasalazine; thiazinecarboxamides such as ampiroxicam, droxicam, isoxicam, lomoxicam, piroxicam, and tenoxicam, cyclooxygenase-II inhibitors (“COX-II”) such as Celecoxib, Vioxx, Relafen, and Lodine; and others such as epsilon-acetamidocaproic acid, s-adenosylmethionine, 3-amino-4-hydroxybutytic acid, amixetrine, bendazac, benzydamine, .alpha.-bisabolol, bucololome, difenpiramide, ditazol, emorfazone, fepradinol, guaiazulene, nabumetone, nimesulide, oxaceprol, paranyline, perisoxal, proquazone, tenidap and zilenton, or a salt, hydrate, ester, amide, enantiomer, isomer, tautomer, polymorph, or prodrug thereof.

Proton pump inhibitors as used in this specification include, but are not limited to omeprazole, hydroxyomeprazole, esomeprazole, tenatoprazole, lansoprazole, pantoprazole, rabeprazole, dontoprazole, habeprazole, periprazole, ransoprazole, pariprazole, and leminoprazole or a salt, hydrate, ester, amide, enantiomer, isomer, tautomer, polymorph, or prodrug thereof.

H₂-blockers as used in this specification include, but are not limited to cimetidine; ranitidine; ebrotidine; pabutidine; lafutidine; loxtidine and famotidine, or a salt, hydrate, ester, amide, enantiomer, isomer, tautomer, polymorph, or prodrug thereof.

Prostaglandin analogue compounds as used in this specification include, but are not limited to carboprost, tromethamine, dinoprost, tromethamine, dinoprostone, lipoprost, gemeprost, metenoprost, sulprostone, tiaprost and misoprostol, or a salt, hydrate, ester, amide, enantiomer, isomer, tautomer, polymorph, or prodrug thereof.

The tem “active agent”, “active ingredient”, “drug”, and “pharmaceutically active agent” are used interchangeably in the present specification, and refer to a compound that, when administered to a mammal or a human induces a pharmacological effect.

The term “acid-labile pharmaceutical agent” as used in this specification refers to any pharmacologically active drug subject to acid catalyzed degradation.

The term “drug absorption” or “absorption” as used in this specification, refers to the process of movement from the site of administration of a drug toward the systemic circulation, e.g., into the bloodstream of a subject.

The term “prevent” or “prevention” as used in this specification in the context of a gastric acid related disorder means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease. The term “prevent” or “prevention” as used in this specification in the context of an inflammatory disorder means no inflammatory disorder or disease development if none had yet occurred, or no further inflammatory disorder or disease if there had already been development of the inflammatory disorder. Also considered is the ability of one to prevent some or all of the symptoms associated with the inflammatory disorder.

The term “treat” or “treatment” as used in this specification in the context of a gastric acid related disorder refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. “Treat” or “treatment” as used in the context of an inflammatory disorder refers to any treatment of a disorder or disease associated with an inflammatory disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent”.

The term “therapeutically effective amount” or “effective amount” as used in this specification refers to that amount of a pharmaceutical agent to achieve a pharmacological effect. The term “therapeutically effective amount” includes, for example, a prophylactically effective amount. An “effective amount” of a proton pump inhibitor, or an H₂-blocker, is an amount effective to achieve a desired pharmacologic effect or therapeutic improvement without undue adverse side effects. For example, an effective amount of a proton pump inhibitor or an H₂-blocker refers to an amount of proton pump inhibitor or H₂-blocker that reduces acid secretion, or raises gastrointestinal fluid pH, or reduces gastrointestinal bleeding, or reduces the need for blood transfusion, or improves survival rate, or provides for a more rapid recovery from a gastric acid related disorder. An “effective amount” of a nonsteroidal anti-inflammatory drug is an amount effective to achieve a desired pharmacological effect on the subject's condition, without undue adverse side effects. The effective amount of a pharmaceutical agent will be selected by those skilled in the art depending on the particular patient and the disease level. It is understood that “an effective amount” or “a therapeutically effective amount” can vary from subject to subject, due to variation in metabolism of therapeutic agents such as prostaglandin analogue compounds, proton pump inhibitors, H2-blockers and/or nonsteroidal anti-inflammatory agents, age, weight, general condition of the subject, the condition being treated, the severity of the condition being treated, and the judgment of the prescribing physician.

The term “pharmaceutically acceptable” salt, ester or other derivative of an active agent as used in the present specification is a salt, ester or other derivative which is not biologically or otherwise undesirable. Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). Other derivatives and analogs of the active agents may be prepared using standard techniques known to those skilled in the art of synthetic organic chemistry, or may be deduced by reference to the pertinent literature.

The term “dosage form” as used in the present specification refers to a single entity for drug administration. Non-limiting examples include a single tablet or capsule comprising the NSAID comprising granules or a single tablet or capsule combining both the NSAID comprising granules and/or a prostaglandin analogue compound, and/or a proton pump inhibitor and or an H₂-blocker.

The terms “anti-adherents,” “glidants,” or “anti-adhesion” agents” as used in the present specification, prevent components of the formulation from aggregating or sticking and improve flow characteristics of a material. Non-limiting examples include colloidal silicon dioxide such as Cab-o-sil.RTM.; tribasic calcium phosphate, talc, corn starch, DL-leucine, sodium lauryl sulfate, magnesium stearate, calcium stearate, sodium stearate, kaolin, and micronized amorphous silicon dioxide (Syloid.RTM.) and the like.

The term “binders” as used in this specification refers to agents that impart cohesive qualities non-limiting examples of which include alginic acid and salts thereof; cellulose derivatives such as carboxymethylcellulose, methylcellulose (e.g., Methocel.RTM.), hydroxypropylmethylcellulose, hydroxyethylcellulose, hydroxypropylcellulose (e.g., Klucel.RTM.), ethylcellulose (e.g., Ethocel.RTM.), and microcrystalline cellulose (e.g., Avicel.RTM.); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac.RTM.), glucose, dextrose, molasses, mannitol, sorbitol, xylitol (e.g., Xylitab.RTM.), and lactose; a natural or synthetic gum such as acacia, tragacanth, ghatti gum, mucilage of isapol husks, polyvinylpyrrolidone (e.g., Polyvidone.RTM. CL, Kollidon.RTM. CL, Polyplasdone.RTM. XL-10), larch arabogalactan, Veegum.RTM., polyethylene glycol, waxes, and sodium alginate.

The term “carrier materials” as used in this specification refers to any commonly used excipients in pharmaceutics and should be selected on the basis of compatibility with the active ingredient. Non-limiting examples include binders, suspending agents, disintegration agents, filling agents, surfactants, solubilizers, stabilizers, lubricants, wetting agents, diluents, and the like. Pharmaceutically compatible carrier material may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. [See Remington: The Science and Practice of Pharmacy, Nineteenth Ed (Easton, Pa.: Mack Publishing Company, 1995); Hoover, John E., Remington 's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999)].

The term “diluents” as used in this specification refers to agents that increase bulk of the composition to facilitate compression, non-limiting examples of which include lactose, starch, mannitol, sorbitol, dextrose, microcrystalline cellulose such as Avicel.RTM., dibasic calcium phosphate, dicalcium phosphate dihydrate, tricalcium phosphate, calcium phosphate, anhydrous lactose, spray-dried lactose, pregelatinzed starch, compressible sugar, such as Di-Pac.RT,. (Amstar), mannitol; hydroxypropylmethylcellulose, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonites, and the like.

The term “disintegration agents” as used in this specification refers to agents that facilitate the breakup or disintegration of a substance. Non-limiting examples include a starch, e.g., a natural starch such as corn starch or potato starch, a pregelatinized starch such as National 1551 or Amijel.RTM., or sodium starch glycolate such as Promogel.RTM. or Explotab.RTM., a cellulose such as a wood product, methylcrystalline cellulose, e.g., Avicel.RTM., Avicel.RTM. PH101, Avicel.RTM. PH102, Avicel.RTM. PH105, Elcema.RTM. P100, Emcocel.RTM., Vivacel.RTM., Ming Tia.RTM., and Solka-Floc.RTM., methylcellulose, croscarmellose, or a cross-linked cellulose, such as cross-linked sodium carboxymethylcellulose (Ac-Di-Sol.RTM.), cross-linked carboxymethylcellulose, or cross-linked croscarmellose, a cross-linked starch such as sodium starch glycolate, a cross-linked polymer such as crospovidone, a cross-linked polyvinylpyrrolidone, an alginate such as alginic acid or a salt of alginic acid such as sodium alginate, a clay such as Veegum.RTM. HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

The term “filling agents” as used in this specification, include, but are not limited to lactose, calcium carbonate, calcium phosphate, dibasic calcium phosphate, calcium sulfate, microcrystalline cellulose, cellulose powder, dextrose; dextrates; dextran, starches, pregelatinized starch, sucrose, xylitol, lactitol, mannitol, sorbitol, sodium chloride, polyethylene glycol, and the like.

The term “lubricants” as used in this specification refers to agents that prevent, reduce or inhibit adhesion or friction of materials. Non-limiting examples include stearic acid, calcium hydroxide, talc, sodium stearyl fumerate, a hydrocarbon such as mineral oil, or hydrogenated vegetable oil such as hydrogenated soybean oil (Sterotex.RTM.), higher fatty acids and their alkali-metal and alkaline earth metal salts, such as aluminum, calcium, magnesium, zinc, stearic acid, sodium stearates, glycerol, talc, waxes, Stearowet.RTM., boric acid, sodium benzoate, sodium acetate, sodium chloride, leucine, a polyethylene glycol or a methoxypolyethylene glycol such as Carbowax.TM., sodium oleate, glyceryl behenate, polyethylene glycol, magnesium or sodium lauryl sulfate, colloidal silica such as Syloid.TM., Carb-O-Sil.RTM., a starch such as corn starch, silicone oil, a surfactant, and the like.

The present invention relates to a novel multi-layer oral dosage form comprising a first layer which can essentially behave as an enteric coated dosage form without the need for the formulation and application of an enteric coating, and a second layer formulated for immediate release of a prostaglandin analogue compound, and/or a proton pump inhibitor and/or an H₂-blocker. In an embodiment, the novel multi-layer oral dosage form is a bi-layer oral dosage form. Although the dosage form can transport a whole range of active ingredients, the dosage form is particularly suitable for products containing non-steroidal anti-inflammatory drugs, prostaglandin analogue compounds, and/or a proton pump inhibitors and/or an H₂-blockers.

In accordance with the present invention, the oral bi-layer dosage forms are characterized by having a first layer exhibiting high stability in acidic media and exhibiting a rapid release of active ingredient(s) (i.e. NSAIDs) at increased pH. The release can be controlled by the size and composition of the granules. The method for manufacturing the oral dosage forms of the present invention, more specifically the first layer exhibiting delayed release characteristics, is both simple and cost efficient compared to the manufacture of conventional enterically-coated products.

In an embodiment, the present invention relates to oral dosage forms, i.e. granules comprising one or more NSAIDs which are tableted into oral bi-layer dosage forms. Even though the active ingredient to be formulated into the granules is preferably a NSAID(s), it is well within the capacity of a skilled technician to provide other active ingredients. The active ingredient is substantially uniformly distributed in a composition comprising one or more excipients selected from the group consisting of solid aliphatic alcohols, mixtures of esters of saturated fatty alcohols and saturated fatty acids or natural or synthetic waxes, hydrogenated castor oil, hydrogenated vegetable oil, gums, or mixtures thereof, and one or more acrylic and/or methacrylic acid polymers and/or copolymers exhibiting a pH-dependent solubility. It was discovered that pH-sensitive polymers, particularly methacrylate-based polymers, more particularly anionic polymers containing carboxylic and/or carboxylate functions, may be included in the melt granulation process.

In an embodiment, the present invention relates to particularly advantageous preparations that can be obtained by adding polymers and/or copolymers exhibiting a pH-dependent solubility to a molten fatty alcohol and the use of such mixtures for granulating the active ingredient.

In an embodiment of the present invention, the molten fatty alcohol is cetyl alcohol. The quality and composition of the cetyl alcohol (i.e. its content in C-14, C-16 and C-18 fractions), more specifically the level of stearyl alcohol (C18) contained therein, has a direct influence on the coating/granulation process. The stearyl alcohol content has a direct influence on the melting point of the wax (i.e. cetyl alcohol). It is important that the melting point of the fatty alcohol (i.e. cetyl alcohol) remain within the range at which the melt granulation is to be performed i.e. from about 50 to about 65° C. The composition of the cetyl alcohol (i. e. its content in C-14, C-16 and C-18 fractions) has a direct impact on the coating process. An adequate cetyl alcohol composition ensures that all of the active ingredient particles are properly embedded in the wax/pH sensitive polymer mix. In an embodiment of the present invention, a cetyl alcohol having a C-16 fraction of not less than 95% and a stearyl alcohol content ranging from about 1-5% is used in order to ensure adequate protection of the active ingredient and to avoid premature release of the active in the stomach. The remaining content is made-up of a C-14 fraction (myristyl alcohol). Non-limiting examples of cetyl alcohols that are within the spirit of the present invention include Aarhus Olie (C-14=0.3%; C-16=97.0%; C-18=2.6%), A&C Chemicals (C-14=0.2%; C-16=98.8%; C-18=0.8%), and Crodacol C-95 (C-14=2.5%; C-16=95.0%; C-18=1.5%). The determination of other adequate cetyl alcohol waxes is well within the capacity of a skilled technician.

The melt granulation is conducted in a suitable temperature range, commonly between about 50° C. to about 65° C., controlled mixing, and controlled cooling. The controlled mixing and cooling of the wet mass is critical to achieving a desired particle size distribution for the final granules. If the particle size distribution is not adequate, the mix can be reheated to about 65° C. and the mixing and cooling process can be repeated.

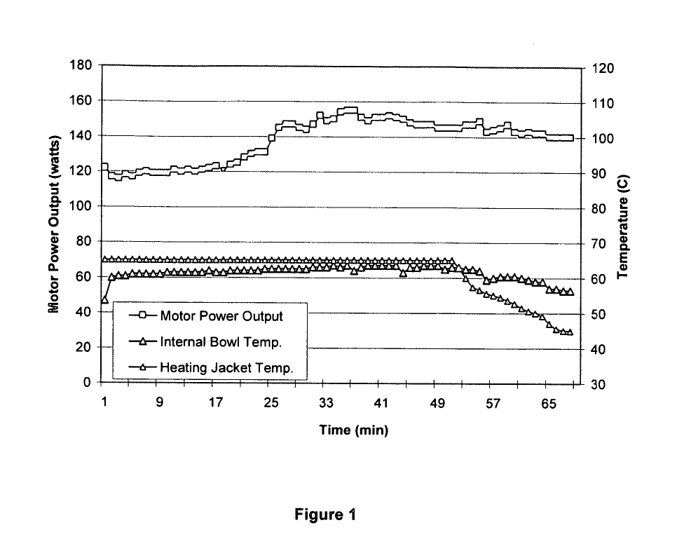

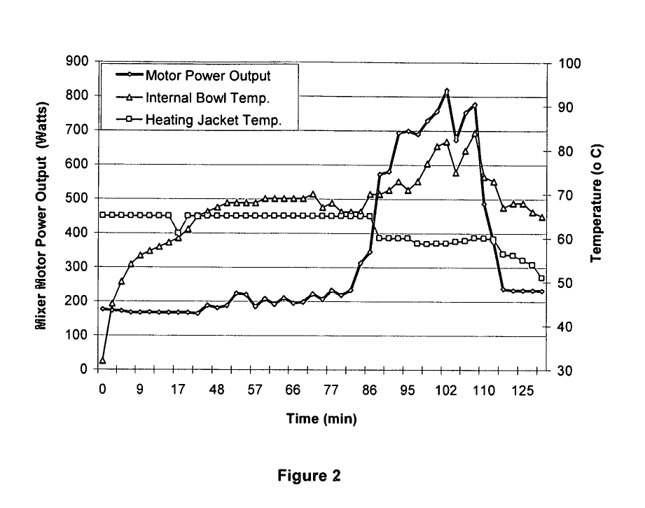

The above described melt granulation procedure provides for substantially homogeneous waxy granules comprising a pH-dependent component in which the active (i.e. NSAID) is entrapped. The hydrophobic character of the waxy component and the nature of the pH-dependent component concomitantly ensure that the active agent remains substantially undissolved during its passage through the stomach. However, at higher pH values such as encountered in the intestine, the pH-dependent component (i.e. polymer) becomes soluble, resulting in the formation of pores in the waxy granules and allowing for the active to be released to the medium. The mixer motor power output is indicative of the progress of the coating process. The wattage jump (FIGS. 1 and 2) is indicative of the absence of any “dry” powder particles, i.e. particles of active agent that are not coated by the wax/pH sensitive polymer mix. This transition is characterized by a sudden increase in the shear resistance of the wet mix, which is reflected in a higher power uptake. Once the full coating of the active particles has been achieved, the shear resistance will remain constant and will depend only on the temperature.

It is important to determine the correct endpoint of the coating process in order to ensure that all of the active ingredient particles are properly embedded in the wax/pH-sensitive polymer mix. If the granulation endpoint is not reached, a portion of the active ingredient will be insufficiently coated. This will inherently result in a premature dissolution of the active ingredient.

The prepared granules are advantageously passed through appropriate sieves in order to obtain granules having a diameter of less than about 850 microns.

Additional pharmaceutically acceptable excipients may be added to improve binding, disintegration and lubrification and are within the capacity of a skilled technician.

In an embodiment, the present invention relates to preparations in which: i) one or more active ingredient(s) is/are substantially uniformly distributed in a mass composed of a mixture comprising at least one saturated fatty alcohol and at least one pH-dependent soluble polymeric binder powder; ii) the procedure consists in homogeneously granulating the active ingredient(s) and the polymeric binder powder(s) and mixing with a molten fatty alcohol; iii) the mixture is slowly cooled down at a rate not exceeding 1° C./min; iv) the resulting preparation is passed through appropriate sieves in order to obtain granules having a diameter of less than about 1000 microns and preferably less than about 850 microns; v) the obtained granulated material can be used in association with one or more additional active ingredients (i.e. prostaglandin analogue compounds, and/or a proton pump inhibitors and/or an H₂-blockers), and/or an external excipient phase consisting of fillers, binders, disintegrants, adjuvants, etc. to obtain tablets. In a particular embodiment, the active ingredient in an NSAID.

In a further embodiment, the present invention relates to a pharmaceutical oral dosage form taking the form of a multi-layer tablet. The multi-layer tablet includes a layer comprising one or more NSAIDs, which layer is prepared by the above-described melt granulation procedure. The multi-layer tablet further includes a layer comprising one or more prostaglandin analogue compounds, and/or one or more proton pump inhibitors and/or one or more H₂-blockers. The tablet may optionally further include a separating layer between the layers containing the active ingredients. Such a multi-layer tablet would decrease the risk of the development and/or exacerbation of ulcers which may occur during NSAID therapy. Moreover, such a multi-layer tablet provides for the prophylactic treatment of a patient who is on NSAID therapy in order to minimize gastrointestinal side-effects. In a particular embodiment, the pharmaceutical oral dosage form is a bi-layer tablet comprising diclofenac and misoprostol or physiologically acceptable salts thereof.

In a further embodiment, the present invention relates to a method for manufacturing a multi-layer tablet comprising a layer providing for the delayed release of one or more NSAIDs, and a further layer providing for the immediate release of a prostaglandin analogue compound, and/or a proton pump inhibitor, and/or an H₂-blocker. The delayed release layer is manufactured using a novel melt granulation procedure. Using a wax/pH-sensitive polymer mix, a gastric insoluble composition is obtained in which the active ingredient is entrapped, ensuring its delayed release. During the melt granulation process, the active ingredient particles are fully embedded in the wax/pH-sensitive polymer mix. A wattage jump in the mixer motor output is indicative of the active ingredient particles being fully coated by the wax/pH-sensitive polymer mix. Contrary to conventional enteric coating procedures wherein particles, granules or beads comprising an active are coated using a polymeric solution or dispersion, the melt granulation procedure of the present invention provides for enterically coated active ingredient containing particles, without an actual enteric coating step. Moreover, contrary to the classical melt granulation procedures known in the art, the active-containing admixtures of the present invention, optionally comprising suitable excipients, provide for a granulated active agent that can be directly compressed or encapsulated together with one or more additional active ingredients.

Light-protective pigments may also be incorporated in the melt granulation process so as to impart light-protective properties to the resulting granules. This provides for the added advantage of avoiding the need for the application of a light-protective coating on the compressed dosage form. Organic or inorganic pigments may be advantageously used in accordance with the present invention. The pigments may, for example, be incorporated in the powder blend or in the granulating liquid solution. Non-limiting examples of inorganic pigments include titanium dioxide, zinc oxide, carbon black, cadmium sulfide, cadmium selenide, chromium oxide, iron oxide, and lead oxide. In an embodiment of the present invention, the light protective pigment is titanium dioxide. Non-limiting examples of organic pigments include azo pigments, anthraquinones, phthalocyanines, tetrachloroisoindolinones, quinacridones, isoindolines, perylenes, and pyrrolopyrroles (such as Pigment Red 254). Other inorganic and organic pigments are known in the art, and are within the capacity of a skilled technician.

In an embodiment, the pharmaceutical oral dosage form of the present invention may further comprise one or more pharmaceutically acceptable excipients selected from the group consisting of binding agents, disintegrants, adhesives and wetting agents. In a further embodiment of the present invention, the pharmaceutical oral dosage form may be in the form of a multiparticulate composition that can be readily compressed into matrix tablets. Moreover, the NSAID comprising multiparticulate composition is particularly resistant to gastric fluid and exhibits an immediate release of drug at pH levels of about 5.5.

In an embodiment, the oral dosage forms of the present invention may optionally further comprise one or more pharmaceutically acceptable binding agents and/or adhesives, particularly for tablet formulations. Suitable binding agents and/or adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and/or adhesives include, either individually or in combination, acacia; been wax; gelatin; glucose; starches such as, but not limited to, pregelatinized starches (e.g., National® 1511 and National® 1500); celluloses such as, but not limited to, methylcellulose; alginic acid and pharmaceutically acceptable salts thereof; PEG; guar gum; polysaccharide acids; povidone (e.g. povidone K-15®, K-30® and K-29132®); ethylcellulose (e.g. Ethocel®); fatty alcohols; fatty acid esters; and natural or synthetic waxes. More preferred binding agents and/or adhesives are selected from the group consisting of fatty alcohols, fatty acid esters, and natural or synthetic waxes. Such binding agents and/or adhesives, if present in the pharmaceutical oral dosage forms of the present invention, constitute in total from about 5% to about 60%, preferably from about 10% to about 60%, and more preferably from about 15% to about 60%, of the total weight of the composition.

In an embodiment, the delayed release properties of the oral dosage forms of the present invention are, at least in part, obtained by using a pH-dependent material such as a pharmaceutically acceptable acrylic polymer, non-limiting examples of which include acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In an embodiment of the present invention, the pH-dependent polymers and/or copolymers constitute from about 5% to about 50% of the total weight of the composition. In a further embodiment of the present invention, the pH-dependent polymers and/or copolymers constitute from about 10% to about 30% of the total weight of the composition. In yet a further embodiment of the present invention, the pH-dependent polymers and/or copolymers constitute from about 12% to about 20% of the total weight of the composition. In order to obtain a desirable dissolution profile of the active(s), it may be necessary to incorporate differing amounts of a variety of methacrylic acid copolymers having differing properties, i.e. having differing amounts of carboxylic acid functional groups capable of being protonated or deprotonated depending on the pH changes.

In an embodiment of the present invention, the pharmaceutically acceptable acrylic polymer is comprised of one or more anionic methacrylate copolymers well known in the art. Such polymers have been described (USP NF XXVII) as being fully polymerized copolymers of acrylic and methacrylic acid esters and as having a certain number of carboxylic acid groups.

Certain methacrylic acid ester-type polymers are generally useful for preparing pH-dependent coatings. For example, the family of copolymers synthesized from diethylaminoethyl methacrylate and other neutral methacrylic esters, also known as methacrylic acid copolymer or polymeric methacrylates (commercially available as Eudragit® RTM from Rohm Pharma) are very suited for preparing pH dependent coatings.

Acrylic coatings comprising a mixture of two acrylic resin lacquers, commercially available from Rohm Pharma under the Trade-names Eudragit.RTM. RL30D® and Eudragit.RTM. RS30D® respectively, are known. Eudragit.RTM. RL30D® and Eudragit.RTM. RS30D® are copolymers of acrylic and methacrylic esters with a low content of quaternary ammonium groups. However, coatings formed from these materials are swellable and permeable in aqueous solutions and digestive fluids.

Eudragit.RTM. RL/RS® dispersions may be mixed together in any desired ratio in order to ultimately obtain a controlled-release formulation having a desirable dissolution profile. Desirable controlled-release formulations may be obtained, for instance, from a retardant coating derived from 100% Eudragit.RTM. RL®, 50% Eudragit.RTM. RL®/50% Eudragit.RTM. RS®, and 10% Eudragit.RTM. RL/Eudragit.RTM. 90% RS. Of course, one skilled in the art will recognize that other acrylic polymers may also be used, such as, for example, Eudragit.RTM. L®.

Other standard enteric coating materials may also be used and are within the capacity of a skilled technician. Non-limiting examples of further such enteric coating materials include phthalates, e.g. cellulose acetate phthalate or preferably hydroxypropylacetate phthalate or polyvinylacetate phthalate. Mixtures of these and other materials may also be used to produce delayed release coatings, and are within the capacity of a skilled technician.

In an embodiment, the oral dosage forms of the present invention may optionally comprise one or more pharmaceutically acceptable disintegrants as further excipients, particularly for tablet formulations. Suitable disintegrants include, either individually or in combination, starches, including sodium starch glycolate (e.g., Explotab® of PenWest) and pregelatinized corn starches (e.g., National® 1551, National® 1550, and Colorcon® 1500); celluloses such as purified cellulose, microcrystalline cellulose, methylcellulose, carboxymethylcellulose and sodium carboxymethylcellulose; crosscarmellose sodium (e.g., Ac-Di-Sol® of FMC); alginates; crospovidone; and gums such as agar and guar. In an embodiment of the present invention, the disintegrant is crosscarmelose. In a further embodiment, the crosscarmelose is present in an amount ranging from about 5% to about 30% of the total weight of the composition. In a further embodiment, the crosscarmelose is present in an amount ranging from about 7% to about 25% of the total weight of the composition. In yet a further embodiment, the crosscarmelose is present in an amount ranging from about 8% to about 20% of the total weight of the composition.

The oral dosage forms of the present invention may be in the form of tablets or other forms such as for example granule-containing capsules. The tablets may be optionally film coated with suitable coatings such as anti-sticking coatings or color coatings.

The melt-granulation process of the present invention can accommodate a vast number of pharmaceutically active molecules and provides for preparations having advantageous flowing, tabletting, disintegration and dissolution properties. Moreover, the melt-granulation process of the present invention provides for pharmaceutical preparations that can be conveniently combined with a further formulation to provide a bi-layer tablet upon compression.

Examples of Tablet Design

A bi-layer oral dosage form comprising a first layer having delayed release characteristics and a second layer having immediate release characteristics is described. The delayed release formulation can essentially behave as an enteric coated dosage form without the need for the formulation and application of an enteric coating. The immediate release formulation was prepared by dry-mixing of the active with pharmaceutically acceptable excipients. The immediate release layer may be formulated using further procedures and pharmaceutical excipients well known to those of ordinary skill in the art.

Two different approaches, both based on the melt-granulation process as described hereinabove, were considered for the preparation of the delayed release formulation: (i) a first approach with delayed release characteristics being designed at granule level; and (ii) a second approach with delayed release characteristics being designed at the NSAID comprising tablet layer level.

### EXAMPLE 1

Gastro-Resistant Granule Preparation

The active ingredient (Diclofenac-Na) and the polymer (Eudragit L100), in a 1:1 ratio, are placed in a jacketed bowl (i.e. mixer bowl) and mixed for homogenization. The jacket temperature is kept at about 65° C., the motor output is kept at about 120-121 watts, and the chopper speed is set to about 1700 rpm. The chopper speed and the blade speed both depend on the size and filling weight of the bowl. Representative impeller and chopper speeds as a function of bowl capacity are provided hereinbelow in Table 1. Generally, the blade speed has to be optimized to ensure proper mixing of the liquid wax and the powder blend, whereas the chopper speed is responsible for the proper particle size of the resulting granules.

The jacket temperature is kept above the melting point range of the wax, more particularly about 10° C. above the melting point range of the wax. The granulation liquid is obtained by heating the fatty alcohol to about 55° C. The liquefied (molten) fatty alcohol is slowly added in portions to the preheated mixed powder blend. In an embodiment of the present invention, the fatty alcohol is cetyl alcohol. In a further embodiment, a cetyl alcohol having a C-16 fraction of not less than 95% and a stearyl alcohol content (C-18) ranging from about 1-5% is used in order to ensure sufficient protection of the active ingredient in order to avoid premature release in the stomach. The remaining content is made-up of a C14 fraction (myristyl alcohol).

The internal bowl temperature is continuously monitored and rises slowly to about 63° C. Upon further adding molten fatty alcohol in small portions, the power output of the motor will start increasing at a steeper rate to approximately 150 watts and will eventually level off. This leveling off is indicative of having reached the endpoint of the coating process, the diclofenac-Na particles now being substantially fully coated by molten wax. The wet mass is continued to be stirred at the above conditions and the mass is then allowed to slowly cool down at a rate not exceeding about 1° C./min. The cooling process has to occur in a controlled fashion and must occur slowly enough so as to prevent agglomeration of the material. It was observed that a cooling rate of about 1° C./min provided for optimal results, avoiding agglomeration of the material. When the temperature of the mix reaches about 52° C., the chopper speed is reduced to about 1500 rpm. The chopper speed at this point is critical in order to achieve the desired granule particle size distribution (i.e. about 60% of granules between 400- 800 □m). If the chopper speed is too low, agglomeration tends to occur.

The blend is further mixed until the temperature reaches about 45° C., at which point any further cooling does not have to be in accordance with a prescribed cooling rate (i.e. the temperature of the mix has dropped below the solidification point of the wax). Controlled cooling has to occur until the temperature of the mix has dropped sufficiently below the solidification point the fatty alcohol. Generally, a controlled cooling to about 5-10° C. below the solidification point of the fatty alcohol is sufficient. The granulated material was then transferred to a metal tray, and cooled to about 22-24° C. The mixer motor output, the internal bowl temperature and the heating jacket temperature as a function of time for a diclofenac-Na granulation, as performed in a 1 L bowl, is illustrated in FIG. 1. In the present example, the impeller speed was set at 800 rpm and the chopper speed was set at 1700 rpm. The mixer motor output, the internal bowl temperature and the heating jacket temperature as a function of time for a diclofenac-Na granulation scale-up trial, as performed in a 6 L bowl, is illustrated in FIG. 2. The leveling off of the mixer motor power output is indicative of the diclofenac-Na being fully coated. In the case of the 6 L bowl, the impeller speed was set at 615 rpm and the chopper speed was set at 1500 rpm.

An appropriate grinder was then used to mill the granulated material. The milled material was then screened through a 2 mm and then through a 0.850 mm screen.

TABLE 1
Impeller and chopper speeds as a function of bowl capacity.

| | Impeller | |
|---|---|---|
| Bowl Capacity (L) | Speed (rpm) | Chopper Speed (rpm) |
| 1 | 800 | 1700 |
| 6 | 615; | 1500 for 100 minutes; |
| | 300 | Increased to 1600 for 23 minutes; |
| | | 1700 |

### EXAMPLE 2

A. NSAID Comprising Delayed Release Layer Properties are Ensured at Granule Level: Multiparticulates Having Delayed Release Characteristics are Obtained From a Formulation in Accordance With an Embodiment of the Present Invention

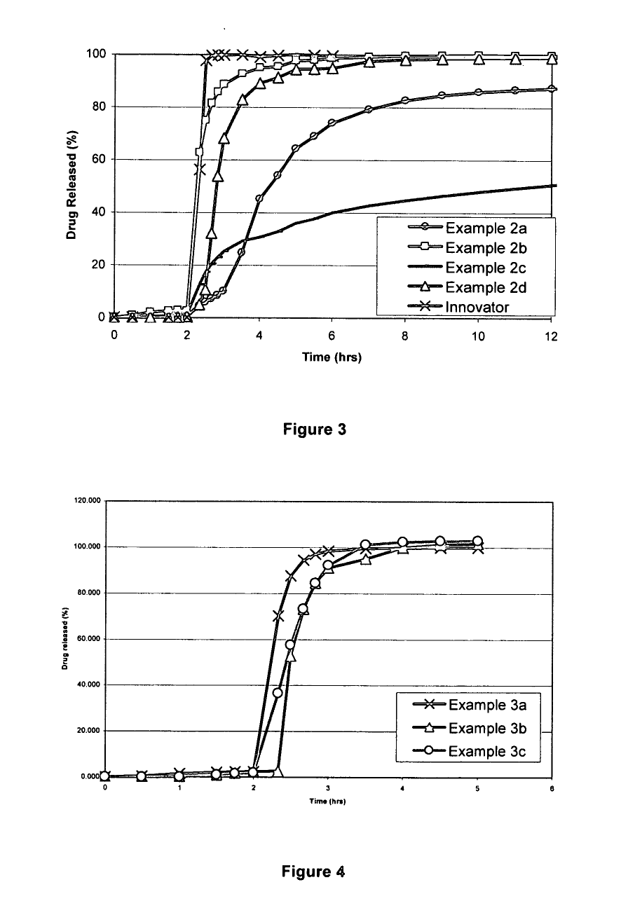

The granules as obtained following the procedure of Example 1 were mixed with an appropriate amount of disintegrant, and the resultant composition was compressed into tablets having a weight of about 625 mg and a diameter of about 8.5 mm using a single punch press. The final formulation expressed as weight percentages contained about 80% granulates (composition breakdown: 40% active, 50% fatty alcohol and 10% methacrylic polymer) and 20% disintegrant (Table 2: 2b). The composition of further formulations, comprising from about 215 mg to about 250 mg of diclofenac, are also illustrated hereinbelow in Table 2 (2a, 2c, 2d). The tablets were then subjected to dissolution testing (USP apparatus II, 200 rpm, 2 h SGF, 2 h SIF) as illustrated in FIG. 3.

TABLE 2
Composition of diclofenac comprising delayed release granules.

| Ingredients | Example 2a | Example 2b | Example 2c | Example 2d |
|---|---|---|---|---|
| Diclofenac | 92% | 80% | 80% | 80% |
| granules | | | | |
| Disintegrant | | | | |
| Type A¹ | 8% | 20% | — | — |
| Type B² | — | — | 20% | — |
| Type C³ | — | — | — | 20% |

¹Cross-linked sodium carboxymethyl cellulose (crosscarmelose);
²Crospovidone;
³Cross-linked carboxymethyl starch (Explotab).

### EXAMPLE 3

Diclofenac-Na and a fraction of polymer (30% of the total amount of polymer) were placed in a bowl and mixed for homogenization. Dissolution of the fatty alcohol in ethanol, together with a second fraction of polymer (70% of the total amount of polymer), provided the granulation liquid. The granulation process was conducted without a heating jacket, a mixer speed of about 500 rpm and a chopper speed of about 1200 rpm until granulation occurred. The granulated material was then transferred and the agglomerates broken down by any suitable means, which will comminute oversized agglomerates and produce a mixture of powder and small particles preferably with a diameter of under about 0.85 mm. An appropriate amount of disintegrant was added and the resultant mixture was compressed into tablets. The tablets of Example 3 were then subjected to three dissolution tests, illustrated by Examples 3a, 3b and 3c, in accordance with USP apparatus II, 200 rpm, 2 h SGF, 2 h SIF.

The dissolution profiles 3a, 3b and 3c, as shown in FIG. 4, indicate a modulation of the rate of release of diclofenac-Na over time in simulated intestinal Fluid (SIF). The rate of release is dependent on the ratio of active (diclofenac-Na) to pH-dependent polymer.

The disintegrant type and proportion were kept constant in all three preparations; the composition of active principle, aliphatic alcohol and pH-dependent polymer in the granules was varied as shown below in Table 3.

TABLE 3
Granule composition

| Ingredients | Example 3a | Example 3b | Example 3c |
|---|---|---|---|
| Active principle | 41.3 | 41.0 | 33.0 |
| Aliphatic alcohol | 12.2 | 18.0 | 23.3 |
| (binder) | | | |
| pH-dependent | 46.5 | 41.0 | 43.7 |
| polymer | | | |
| Final tablet (mg) | +8% | +8% | +8% |
| | disintegrant B | disintegrant B | disintegrant B |
| | 200 mg | 200 mg | 250 mg |

B. Delayed Release Properties are Ensured by NSAID Comprising Tablet Layer.

In an embodiment of the present invention, a composition having delayed release characteristics is obtained by a melt granulation process. Such composition provides for protection of the active principle(s) during the passage through the gastric segment by limiting the capacity of the tablet to hydrate in the acidic medium. Thus, by essentially maintaining its integrity, the tablet is able to ensure delayed release characteristics.

### EXAMPLE 4

The fatty alcohol (wax) is first melted. Diclofenac-Na and a methacrylic copolymer are placed in a jacketed bowl and mixed for homogenization. The jacket temperature is kept at about 65° C., the motor output is kept at about 120-121 watts, and the chopper speed is set to about 1700 rpm. The jacket temperature is kept above the melting point range of the wax, more particularly about 10° C. above the melting point range of the wax. The granulation liquid is obtained by heating the fatty alcohol to about 55° C. The liquefied (molten) fatty alcohol is slowly added in portions to the preheated mixed powder blend.

The internal bowl temperature is continuously monitored and rises slowly to 63° C. Upon further adding molten fatty alcohol in small portions, the motor output of the motor will start increasing at a steeper rate to approximately 150 watts and will eventually level off. This leveling off is indicative of having reached the endpoint of the coating process, the diclofenac-Na particles now being substantially fully coated by molten wax. The wet mass is continued to be stirred at the above conditions and the mass is then allowed to slowly cool down at a rate not exceeding 1° C./min. The cooling process has to occur in a controlled fashion and must occur slow enough so as to prevent agglomeration of the material. It was observed that a cooling rate of about 1° C./min provided for optimal results, avoiding agglomeration of the material. When the temperature of the mix reaches about 52° C., the chopper speed is reduced to about 1500 rpm. The blend is further mixed until the temperature reaches about 45° C., at which point any further cooling does not have to be in accordance with a prescribed cooling rate (i.e. the temperature of the mix has dropped below the solidification point of the wax). Controlled cooling has to occur until the temperature of the mix has dropped sufficiently below the solidification point the fatty alcohol. Generally, a controlled cooling to about 5-10° C. below the solidification point of the fatty alcohol is sufficient. The granulated material was then transferred to a metal tray, and cooled to about 22-24° C.

A suitable grinder was then used to mill the granulated material. The milled material was then screened through a 2 mm and then through a 0.850 mm screen. The granulates were then compressed into tablets and subjected to dissolution testing (USP apparatus II, 200 rpm, 2 h SGF, 2 h SIF). The delayed released properties are more pronounced when the matrix approach is used, the dissolution profiles showing a complete drug release after more then 10 h.

### EXAMPLE 5

Light Protection of Granules by Incorporation of One or More Pigments or Flakes.

The active ingredient (Diclofenac-Na) and the polymer were placed in a jacketed bowl and mixed for homogenization. The granulating liquid was obtained by adding at least one pigment powder to the fatty alcohol solution, prepared ahead of time using ethanol and heating to about 55° C. to induce melting. The melt granulation procedure as described hereinabove in Example 4 is followed. The granulated material is then transferred to a metal tray, and cooled to about 22-24° C.

A suitable grinder was then used to mill the granulated material. The milled material was then screened through a 2 mm and then through a 0.850 mm screen. A final admixture is obtained by mixing the granulated material comprising the active with a disintegrant. The resultant preparation was compressed into tablets comprising from about 200 to about 300 mg of active (i.e. diclofenac-Na). The amount of disintegrant was selected to represent from about 5 to about 35 wt % of the active ingredient present in the tablet.

### EXAMPLES 6-8

Granules were prepared as described hereinabove in Example 5 using varying amounts of pigment(s). More specifically, titanium dioxide was set at 4 wt %, 8 wt % and 15 wt % based on the total mass of the granules. Mixtures of titanium dioxide and flakes of a suitable colorant (red #40 lake) were also used to provide opacity to the granulated active.

### EXAMPLE 9

Preparation of the misoprostol layer

Commercially available Misoprostol HPMC 1% trituration is dry blended with crospovidone, microcrystalline cellulose, and colloidal silicon dioxide. Hydrogenated castor oil is then added followed by mixing in a high shear mixer at about 300 rpm for 10 minutes. Finally, magnesium stearate is added as lubricant. The resulting powder blend was compressed along with the pre-compressed diclofenac-Na comprising layer to form a bi-layer tablet with a final hardness of about 12 kPa.

Tablet Testing

Desired tap and bulk densities of the granulation are normally from about 0.3 g/ml to about 1.0 g/ml. Tablet friability is preferably less than about 1.0%, more preferably less than 0.8%, and still more preferably less than about 0.5%, in a standard test.

It is well known in the art that several factors influence dissolution of a drug from its carrier into a solvent medium. Such factors include the surface area of the carrier exposed to the solvent medium, the solubility of the drug in the solvent medium, and the driving forces of the saturation concentration of dissolved materials in the solvent medium.

A composition having a dissolution profile in which substantially less than about 5% of the drug contained therein is released in the first two hours following placement in a SGF dissolution medium is considered to be a delayed-release composition. Immediate-release compositions typically release at least about 50% of the drug contained therein in the first hour following placement in a SGF dissolution medium.

In accordance with an embodiment of the present invention, the granules of the present invention release from about 1% to about 3% of the drug contained therein in the first two hours following placement in a SGF dissolution medium, from about 30% to about 70% of the drug contained therein in the first half-hour hour following placement in a SIF dissolution medium, and at least about 90% of the drug contained therein in the first hour following placement in a SIF dissolution medium.

In accordance with another embodiment of the present invention, the granules of the present invention release from about 0.5% to about 2.5% of the drug contained therein in the first two hours following placement in a SGF dissolution medium, from about 60% to about 80% of the drug contained therein in the first half-hour following placement in a SIF dissolution medium, and at least about 95% of the drug contained therein in the first hour following placement in a SIF dissolution medium.

In accordance with another embodiment of the present invention, the granules of the present invention release from about 0.5% to about 1.5% of the drug contained therein in the first two hours following placement in a SGF dissolution medium, from about 75% to about 85% of the drug contained therein in the first half-hour following placement in a SIF dissolution medium, and substantially complete dissolution of the drug contained therein in the first hour following placement in a SIF dissolution medium.

It is to be understood that the invention is not limited in its application to the details of construction and parts as described hereinabove. The invention is capable of other embodiments and of being practiced in various ways. It is also understood that the phraseology or terminology used herein is for the purpose of description and not limitation. Hence, although the present invention has been described hereinabove by way of illustrative embodiments thereof, it can be modified, without departing from the spirit, scope and nature of the subject invention as defined in the appended claims.

1. A delayed release oral dosage form wherein one or more active ingredients, or pharmaceutically acceptable salts thereof, are substantially uniformly distributed in a composition comprising:
a) one or more excipients selected from the group consisting of solid aliphatic alcohols, mixtures of esters of saturated fatty alcohols and saturated fatty acids, natural or synthetic waxes, hydrogenated castor oil, hydrogenated vegetable oil, gums, and mixtures thereof; and
b) one or more polymers and/or copolymers exhibiting a pH-dependent solubility.

2. The delayed release oral dosage form of claim 1, wherein said composition further comprises at least one type of light opacifying pigment in an amount suitable to confer light protective characteristics to the one or more active ingredients contained in the oral dosage form.

3. The delayed release oral dosage form of claim 1, wherein said composition further comprises pharmaceutically acceptable excipients selected from the group consisting of fillers, binders, disintegrants, adjuvants, adhesives, wetting agents, flow agents, plasticizers and mixtures thereof.

4. The delayed release oral dosage form of claim 1, wherein said one or more excipients is a solid aliphatic alcohol.

5. The delayed release oral dosage form of claim 4, wherein the one or more polymers exhibiting a pH-dependent solubility are selected from the group consisting of acrylic acid polymers, methacrylic acid polymers, acrylic and methacrylic acid copolymers, and mixtures thereof.

6. The delayed release oral dosage form of claim 5, wherein the one or more polymers exhibiting a pH-dependent solubility are selected from the group consisting of acrylic acid, methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

7. The delayed release oral dosage form of claim 6, wherein the light opacifying pigment is selected from the group consisting of inorganic pigments and organic pigments.

8. The delayed release oral dosage form of claim 7, wherein the inorganic pigments are selected from the group consisting of titanium dioxide, zinc oxide, carbon black, cadmium sulfide, cadmium selenide, chromium oxide, iron oxide, and lead oxide.

9. The delayed release oral dosage form of claim 7, wherein the organic pigments are selected from the group consisting of azo pigments, anthraquinones, phthalocyanines, tetrachloroisoindolinones, quinacridones, isoindolines, perylenes, and pyrrolopyrroles.

10. The delayed release oral dosage form of claim 8, wherein the inorganic pigment is titanium dioxide.

11. The delayed release oral dosage form of claim 4, wherein the solid aliphatic alcohol is cetyl alcohol.

12. The delayed release oral dosage form of claim 1, wherein said one or more active ingredients is selected from the group consisting of NSAIDs.

13. The delayed release oral dosage form of claim 12, wherein the NSAID is diclofenac.

14. The delayed release oral dosage form of claim 11, wherein the cetyl alcohol comprises a C-16 fraction of at least 95% and a C-18 fraction ranging from about 1 to about 5%.

15. The delayed release oral dosage form of claim 14, further comprising a C-14 fraction.

16. An oral pharmaceutical dosage form, comprising a delayed release formulation including a composition which has substantially uniformly distributed one or more NSAIDs or pharmaceutically acceptable salts thereof, and/or an immediate release formulation of a prostaglandin analogue compound, and/or a proton pump inhibitor (PPI) and/or an H₂-blocker, or pharmaceutically acceptable salts thereof, wherein the composition comprises:
a) one or more excipients selected from the group consisting of solid aliphatic alcohols, mixtures of esters of saturated fatty alcohols and saturated fatty acids, natural or synthetic waxes, hydrogenated castor oil, hydrogenated vegetable oil, gums, and mixtures thereof; and
b) one or more polymers and/or copolymers exhibiting a pH-dependent solubility.

17. The oral pharmaceutical dosage form of claim 16, wherein said delayed release formulation further comprises at least one type of light opacifying pigment in an amount suitable to confer light protective characteristics to the one or more active NSAIDs contained in the oral dosage form.

18. The oral pharmaceutical dosage form of claim 16, wherein said delayed release formulation further comprises pharmaceutically acceptable excipients selected from the group consisting of fillers, binders, disintegrants, adjuvants, adhesives, wetting agents, flow agents, plasticizers and mixtures thereof.

19. The oral pharmaceutical dosage form of claim 16, wherein said one or more excipients is a solid aliphatic alcohol.

20. The oral pharmaceutical dosage form of claim 19, wherein the one or more polymers exhibiting a pH-dependent solubility are selected from the group consisting of acrylic acid polymers, methacrylic acid polymers, acrylic and methacrylic acid copolymers, and mixtures thereof.

21. The oral pharmaceutical dosage form of claim 20, wherein the one or more polymers exhibiting a pH-dependent solubility are selected from the group consisting of acrylic acid, methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

22. The oral pharmaceutical dosage form of claim 21, wherein the light opacifying pigment is selected from the group consisting of inorganic pigments and organic pigments.

23. The oral pharmaceutical dosage form of claim 22, wherein the inorganic pigments are selected from the group consisting of titanium dioxide, zinc oxide, carbon black, cadmium sulfide, cadmium selenide, chromium oxide, iron oxide, and lead oxide.

24. The oral pharmaceutical dosage form of claim 22, wherein the organic pigments are selected from the group consisting of azo pigments, anthraquinones, phthalocyanines, tetrachloroisoindolinones, quinacridones, isoindolines, perylenes, and pyrrolopyrroles.

25. The oral pharmaceutical dosage form of claim 23, wherein the inorganic pigment is titanium dioxide.

26. The oral pharmaceutical dosage form of claim 19, wherein the solid aliphatic alcohol is cetyl alcohol.

27. The oral pharmaceutical dosage form of claim 16 wherein the NSAID is diclofenac.

28. The oral pharmaceutical dosage form of claim 27, wherein the prostaglandin analogue compound and/or proton pump inhibitor (PPI) and/or H₂-blocker is misoprostol.

29. The oral pharmaceutical dosage form of claim 26, wherein the cetyl alcohol comprises a C-16 fraction of at least 95% and a C-18 fraction ranging from about 1 to about 5%.

30. The oral pharmaceutical dosage form of claim 29, further comprising a C-14 fraction.

31. The oral pharmaceutical dosage form of claim 16, comprising a multi-layer tablet.

32. The oral pharmaceutical dosage form of claim 31, wherein the multi-layer tablet is a bi-layer tablet.

33. A process for the manufacture of the delayed release oral dosage form as defined in claim 1 comprising:
a) obtaining through heating a liquid form of said one or more excipients;
b) pre-heating and mixing said one or more active ingredients with said one or more polymers and/or copolymers exhibiting a pH-dependent solubility to obtain a blend; and
c) granulating said blend with said liquid form of said one or more excipients so as to obtain granules.

34. The process of claim 33, wherein said granulating comprises:
d) slowly adding in portions of said liquid form of said one or more excipients to said blend to obtain a wet mass;
e) monitoring the power output of the mixer motor for a leveling off; and
f) slowly cooling the granules at a rate not exceeding 1° C. to obtain a granulated material.

35. The process of claim 34, wherein step (b) further includes mixing with pharmaceutically acceptable excipients selected from the group consisting of fillers, binders, disintegrants, adjuvants, adhesives, wetting agents, flow agents, plasticizers and mixtures thereof.

36. The process of claim 33, wherein the one or more excipients is a solid aliphatic alcohol.

37. The process of claim 36, wherein the one or more polymers and/or copolymers exhibiting a pH-dependent solubility are selected from the group consisting of acrylic acid polymers, methacrylic acid polymers, acrylic and methacrylic acid copolymers, and mixtures thereof.

38. The process of claim 36, wherein the one or more polymers exhibiting a pH-dependent solubility are selected from the group consisting of acrylic acid, methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

39. The process of claim 38, wherein the light opacifying pigment is selected from the group consisting of inorganic pigments and organic pigments.

40. The process of claim 39, wherein the inorganic pigments are selected from the group consisting of titanium dioxide, zinc oxide, carbon black, cadmium sulfide, cadmium selenide, chromium oxide, iron oxide, and lead oxide.

41. The process of claim 39, wherein the organic pigments are selected from the group consisting of azo pigments, anthraquinones, phthalocyanines, tetrachloroisoindolinones, quinacridones, isoindolines, perylenes, and pyrrolopyrroles.

42. The process of claim 40, wherein the inorganic pigment is titanium dioxide.

43. The process of claim 36, wherein the solid aliphatic alcohol is cetyl alcohol.

44. The process of claim 33, wherein said one or more active ingredients is selected from the group consisting of NSAIDs.

45. The process of claim 44, wherein the NSAID is diclofenac.

46. The process of claim 43, wherein the cetyl alcohol comprises a C-16 fraction of at least 95% and a C-18 fraction ranging from about 1 to about 5%.

47. The process of claim 46, further comprising a C-14 fraction.

48. The process of claim 47, wherein the granulated material is ground and compressed into a tablet shape.

49. A process for the manufacture of the oral pharmaceutical dosage form as defined in claims 16 or 17 claim 16 comprising:
a) obtaining through heating a liquid form of said one or more excipients;
b) pre-heating and mixing said one or more active ingredients with said one or more polymers and/or copolymers exhibiting a pH-dependent solubility to obtain a blend; and
c) granulating said blend with said liquid form of said one or more excipients so as to obtain granules.

50. The process of claim 49, wherein said granulating comprises:
d) slowly adding in portions of said liquid form of said one or more excipients to said blend to obtain a wet mass;
e) monitoring the power output of the mixer motor for a leveling off; and
f) slowly cooling the granules at a rate not exceeding 1° C. to obtain a granulated material.

51. The process of claim 50, wherein step (b) further includes mixing with pharmaceutically acceptable excipients selected from the group consisting of fillers, binders, disintegrants, adjuvants, adhesives, wetting agents, flow agents, plasticizers and mixtures thereof.

52. The process of claim 49, wherein the one or more excipients is a solid aliphatic alcohol.

53. The process of claim 52, wherein the one or more polymers and/or copolymers exhibiting a pH-dependent solubility are selected from the group consisting of acrylic acid polymers, methacrylic acid polymers, acrylic and methacrylic acid copolymers, and mixtures thereof.

54. The process of claim 53, wherein the one or more polymers and/or copolymers exhibiting a pH-dependent solubility are selected from the group consisting of acrylic acid, methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

55. The process of claim 54, wherein the light opacifying pigment is selected from the group consisting of inorganic pigments and organic pigments.

56. The process of claim 55, wherein the inorganic pigments are selected from the group consisting of titanium dioxide, zinc oxide, carbon black, cadmium sulfide, cadmium selenide, chromium oxide, iron oxide, and lead oxide.

57. The process of claim 55, wherein the organic pigments are selected from the group consisting of azo pigments, anthraquinones, phthalocyanines, tetrachloroisoindolinones, quinacridones, isoindolines, perylenes, and pyrrolopyrroles.

58. The process of claim 56, wherein the inorganic pigment is titanium dioxide.

59. The process of claim 52, wherein the solid aliphatic alcohol is cetyl alcohol.

60. The process of claim 49, wherein the NSAID is diclofenac.

61. The process of claim 60, wherein the prostaglandin analogue compound and/or proton pump inhibitor and/or H₂-blocker is misoprostol.

62. The process of claim 55, wherein the cetyl alcohol comprises a C-16 fraction of at least 95% and a C-18 fraction ranging from about 1 to about 5%.

63. The process of claim 62, further comprising a C-14 fraction.

64. The process of claim 50, wherein the granulated material is ground and pre-compressed into a first layer.

65. The process of claim 64, wherein the first layer is combined with the immediate release formulation and compressed into a multi-layer tablet shape.

66. The process of claim 65, wherein the multi-layer tablet form is a bi-layer tablet.

67. A method for the treatment of gastrointestinal side-effects associated with NSAID treatment in mammals and humans comprising administering to the host in need thereof a therapeutically effective amount of the dosage form of claim 16.

68. Use of a dosage form in accordance with claim 16, for the manufacture of a medicament for treatment or prevention of gastro-intestinal side-effects associated with NSAID(s) treatment disorders.
